(19) United States
(12) Patent Application Publication
(10) Pub. No.: US 2007/0197977 A1
(43) Pub. Date: Aug. 23, 2007

(54) Disposable syringe

(76) Inventors: Ming-Jeng Shue, Taichung City (TW); Deborah Huang, Taichung City (TW); Phillip Shue, Taichung City (TW)

Correspondence Address: Paul D. Greeley, Esq.;Ohlandt, Greeley, Ruggiero & Perle, L.L.P., 10th Floor, One Landmark Square, Stamford, CT, 06901-2682, US

(21) Appl. No.: 11/451,106

(22) Filed: Jun. 12, 2006

(30) Foreign Application Priority Data
Feb. 22, 2006 (TW) 095105914

(51) Int. Cl. A61M 5/315

(52) U.S. Cl. 604/218

(57) ABSTRACT

A disposable syringe includes a barrel, a needle assembly in fluid communication with the barrel, and a plunger movable in an accommodation chamber of the barrel. The needle assembly includes a needle seat sleeved on a small-diameter wall portion of the barrel, and a tubular insert extending axially into a duct of the needle seat and defining a fluid channel, and a needle cannula in fluid communication with the fluid channel. A space minimizing member is disposed to be in a fluid-tight engagement with an inner surface of the small-diameter wall portion, and has a tubular grip surface which defines a passageway fluidly communicated with the duct such that the insert is in a fluid-tight engagement with the grip surface to thereby communicate the channel with the chamber. Thus, the amount of residual medication fluid in the barrel after completion of an injection operation can be minimized.

### CROSS-REFERENCE TO RELATED APPLICATION

This application claims priority of Taiwanese Application No. 095105914, filed on Feb. 22, 2006.

### BACKGROUND OF THE INVENTION

1. Field of the Invention

This invention relates to a disposable syringe, more particularly to a disposable syringe which minimizes the amount of residual injection fluid in the barrel after completion of an injection operation.

2. Description of the Related Art

Referring to FIG. 1, a conventional disposable syringe 9 is shown to include a barrel 91, a plunger 92, a needle seat 93, and a needle cannula 94. The barrel 91 has small-diameter and large-diameter wall portions 914,913 surrounding an axis to define a passage 912 and an accommodation chamber 911, respectively, a shoulder 915 disposed between the small-diameter and large-diameter wall portions 914,913, and a finger flange 916 formed on a rear end and extending radially. The plunger 92 is slidable in the accommodation chamber 911. The needle seat 93 is air-tightly sleeved on the small-diameter wall portion 914, and has a surrounding wall 933 which surrounds the axis and which defines a needle securing hole 931 and a duct 932. The needle cannula 94 has a secured end 941 secured in the needle securing hole 931, and a tip end 942 extending forwardly of the needle seat 93.

A health care worker can push the plunger 92 forwardly relative to the large-diameter wall portion 913 such that injection fluid in the accommodation chamber 911 is injected into a patient through the passage 912, the duct 932 and the needle cannula 94. The injection operation is completed when the plunger 92 reaches the shoulder 915. However, a relatively large amount of residual injection fluid is trapped in dead chambers of the syringe 9, such as the passage 912 and the duct 932, thereby resulting in medicine waste and aggravated environmental pollution problems.

### SUMMARY OF THE INVENTION

The object of the present invention is to provide a disposable syringe which can minimize the amount of residual injection fluid in the syringe after completion of an injection operation.

According to this invention, the disposable syringe includes a barrel, a tubular needle seat, a tubular insert, a space minimizing member, and a plunger. The barrel has front and rear barrel ends opposite to each other along an axis, and a surrounding barrel wall interconnecting the front and rear barrel ends. The surrounding barrel wall includes small-diameter and large-diameter wall portions which are disposed proximate to the front and rear barrel ends, respectively, and which respectively have front and rear inner wall surfaces defining a passage and an accommodation chamber that are in fluid communication with each other, and a shoulder which is interposed therebetween. The needle seat has a front retaining wall portion which retains a rear secured end of a needle cannula to permit forward extension of a front tip end of the needle cannula from the front retaining wall portion, and a rear sleeve wall portion which defines a duct that is fluidly communicated with the needle cannula, and which is detachably sleeved on the small-diameter wall portion to fluidly communicate the duct with the passage. The tubular insert is disposed to extend from the front retaining wall portion along the axis and into the duct, and has an inner tubular surface which defines a fluid channel that is disposed to be fluidly communicated with the rear secured end of the needle cannula, and an outer tubular surface. The space minimizing member has a tubular abutment surface in a fluid-tight engagement with the front inner wall surface, and a tubular grip surface defining a passageway which fluidly communicates the duct with the accommodation chamber. Thus, when the rear sleeve wall portion is sleeved on the small-diameter wall portion, the tubular insert is inserted into the passage, and is in a fluid-tight engagement with the tubular grip surface to thereby communicate the fluid channel with the accommodation chamber. The plunger is disposed to be movable in the accommodation chamber of the large-diameter wall portion along the axis so as to force a medication fluid in the accommodation chamber to flow into the needle cannula through the passageway and the fluid channel.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a sectional view of a conventional disposable syringe in an injection-completed state;

FIG. 2 is an exploded sectional view of the first preferred embodiment of a disposable syringe according to this invention;

FIG. 3 is a fragmentary sectional view of the first preferred embodiment in an injection-completed state;

FIG. 4 is an exploded sectional view of the second preferred embodiment of a disposable syringe according to this invention;

FIG. 5 is a fragmentary sectional view of the third preferred embodiment in an injection-completed state;

FIG. 6 is a fragmentary sectional view of the third preferred embodiment of a disposable syringe according to this invention;

FIG. 7 is a fragmentary sectional view of the fourth preferred embodiment of a disposable syringe according to this invention;

FIG. 8 is a fragmentary sectional view of the fourth preferred embodiment in a plunger-withdrawn state;

FIG. 9 is a fragmentary sectional view of the fifth preferred embodiment of a disposable syringe according to this invention in a plunger-withdrawn state;

FIG. 10 is an exploded sectional view of the sixth preferred embodiment of a disposable syringe according to this invention;

FIG. 11 is a fragmentary sectional view of the sixth preferred embodiment;

FIG. 12 is a sectional view of the seventh preferred embodiment of a disposable syringe according to this invention;

FIGS. 13-15 are, respectively, fragmentary sectional views of the eighth, ninth and tenth preferred embodiments of a disposable syringe according to this invention in a plunger-withdrawn state; and

FIG. 16 is a fragmentary sectional view of the eleventh preferred embodiment of a disposable syringe according to this invention.

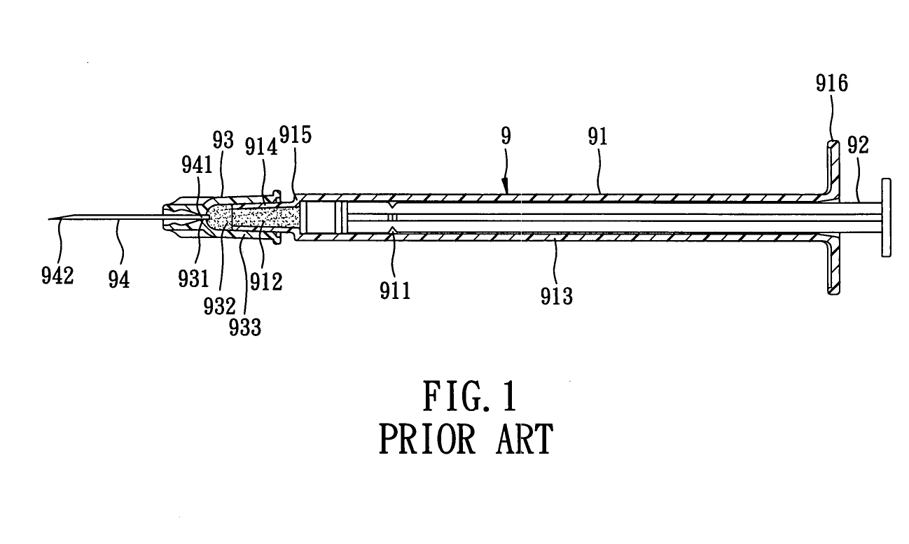

### DETAILED DESCRIPTION OF THE PREFERRED EMBODIMENTS

Before the present invention is described in greater detail, it should be noted that same reference numerals have been used to denote like elements throughout the specification.

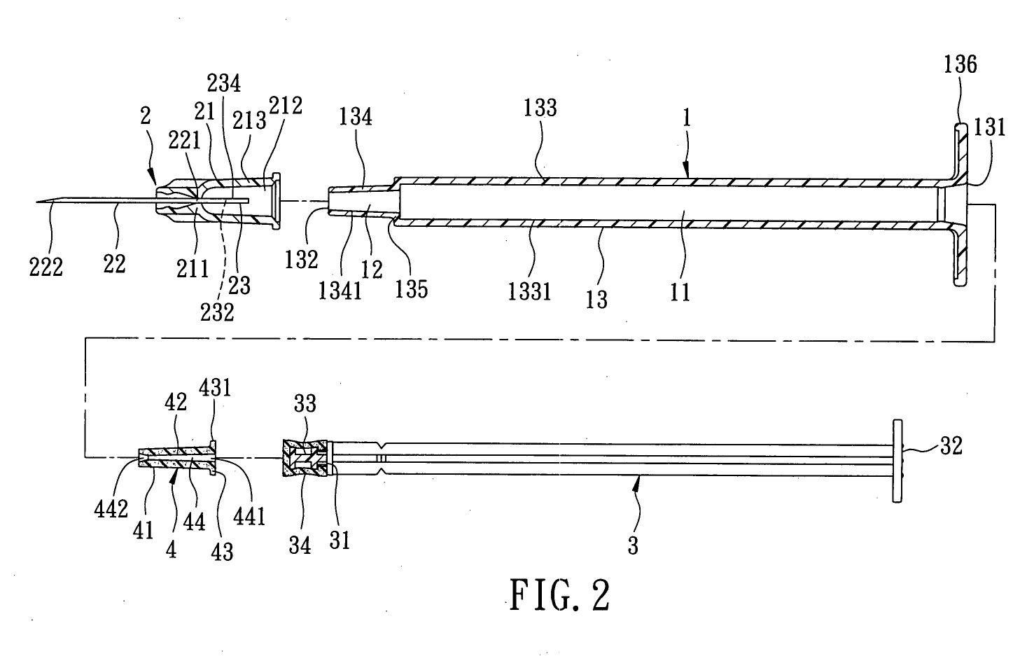

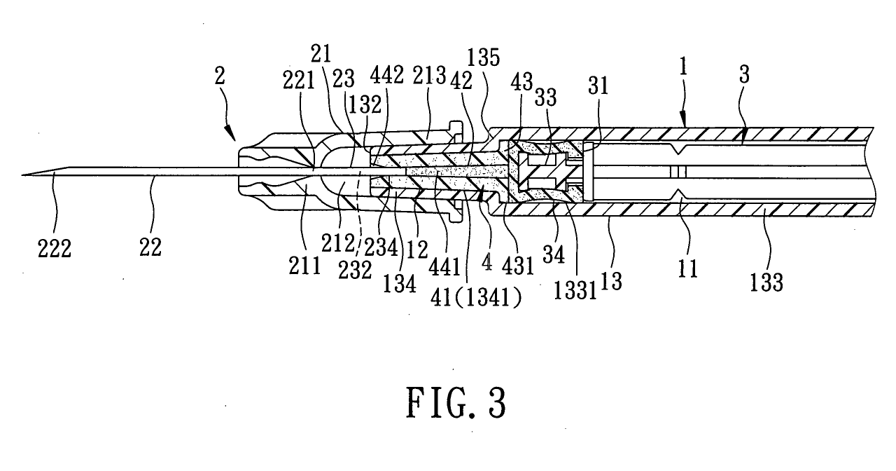

Referring to FIGS. 2 and 3, the first preferred embodiment of a disposable syringe according to the present invention is shown to comprise a barrel 1, a needle assembly 2, a space minimizing member 4, and a plunger 3.

In this embodiment, the barrel 1 is used for receiving medication of a small-volume, e.g. 1 cc. The barrel 1 includes front and rear barrel ends 132,131 opposite to each other along an axis in an axial direction, and a surrounding barrel wall 13 which interconnects the front and rear barrel ends 132,131. The surrounding barrel wall 13 includes small-diameter and large-diameter wall portions 134,133 which are disposed proximate to the front and rear barrel ends 132,131, respectively, and which respectively have front and rear inner wall surfaces 1341,1331 that respectively define a passage 12 and an accommodation chamber 11 which are in fluid communication with each other, a shoulder 135 which is interposed between the small-diameter and large-diameter wall portions 134,133, and a finger flange 136 which extends from the large-diameter wall portion 133 radially and outwardly and which is disposed adjacent to the rear barrel end 131.

The needle assembly 2 includes a needle cannula 22, a tubular needle seat 21, and a tubular insert 23. The needle cannula 22 has a rear secured end 221 and a front tip end 222 opposite to each other along the axis. The tubular needle seat 21 has a front retaining wall portion 211 which retains the rear secured end 221 of the needle cannula 22 to permit forward extension of the front tip end 222 from the front retaining wall portion 211, and a rear sleeve wall portion 213 which is opposite to the front retaining wall portion 211 along the axis to define a duct 212 that is fluidly communicated with the needle cannula 22, and which is detachably sleeved on the small-diameter wall portion 134 to fluidly communicate the duct 212 with the passage 12.

In this embodiment, the tubular insert 23 is integrally formed with the needle cannula 22, and extends rearwardly from the rear secured end 221 along the axis to be disposed into the duct 212. The tubular insert 23 has an inner tubular surface which defines a fluid channel 232 that is disposed to be fluidly communicated with the rear secured end 221 of the needle cannula 22, and an outer tubular surface 234 which is opposite to the inner tubular surface in radial directions relative to the axis.

The space minimizing member 4 has a tubular abutment surface 41 surrounding the axis, a tubular grip surface 42 which is surrounded by the tubular abutment surface 41 and which defines a passageway 44, and a tubular rear barrier flange 43. The space minimizing member 4 is disposed to be inserted into the passage 12 in the small-diameter wall portion 134 from the rear barrel end 131 to bring the tubular abutment surface 41 into fluid-tight abutment against the front inner wall surface 1341. In addition, the tubular rear barrier flange 43 is disposed to abut against the shoulder 135 to prevent forward movement of the space minimizing member 431, and extends radially and outwardly to terminate at an outer flange surface 431. The outer flange surface 431 is in a fluid-tight engagement with the rear inner wall surface 1331. The passageway 44 has a passageway segment 441 which is disposed proximate to the shoulder 135 and which has an inner diameter ranging from about 0.3 to 1.6 mm, and an insert segment 442 which is disposed proximate to the front barrel end 132. Thus, when the rear sleeve wall portion 213 of the needle seat 21 is sleeved on the small-diameter wall portion 134, the tubular insert 23 is inserted into the passageway 44 to bring the outer tubular surface 234 into a fluid-tight engagement with the tubular grip surface 42 to thereby communicate the fluid channel 232 with the accommodation chamber 11.

The plunger 3 is disposed to be movable in the accommodation chamber 11 of the large-diameter wall portion 133 along the axis so as to force a medication fluid in the accommodation chamber 11 to flow into the needle cannula 22 through the passageway 44 and the fluid channel 232. The plunger 3 has a front end wall 31 which confronts the passage 12, a carrier portion 33 which extends from the front end wall 31 forwardly along the axis, a hollow deformable sealing member 34 which is disposed on the carrier portion 33 and which is in fluid-tight and sliding engagement with the rear inner wall surface 1331, and a thumb rest 32 which extends rearwardly and outwardly of the finger flange 136 for being manually operation.

It is noted that by inserting the tubular insert 23 into the passageway 44 and by virtue of the fluid-tight engagement between the outer tubular surface 234 and the tubular grip surface 42, the medication fluid in the accommodation chamber 11 is introduced into the needle cannula 22 directly through the tubular insert 23. Thus, a clearance between the needle cannula 22 and the passage 12 is minimized such that the amount of residual medication fluid in the barrel 1 after completion of an injection operation can be minimized, thereby reducing waste of medication and alleviating environmental pollution.

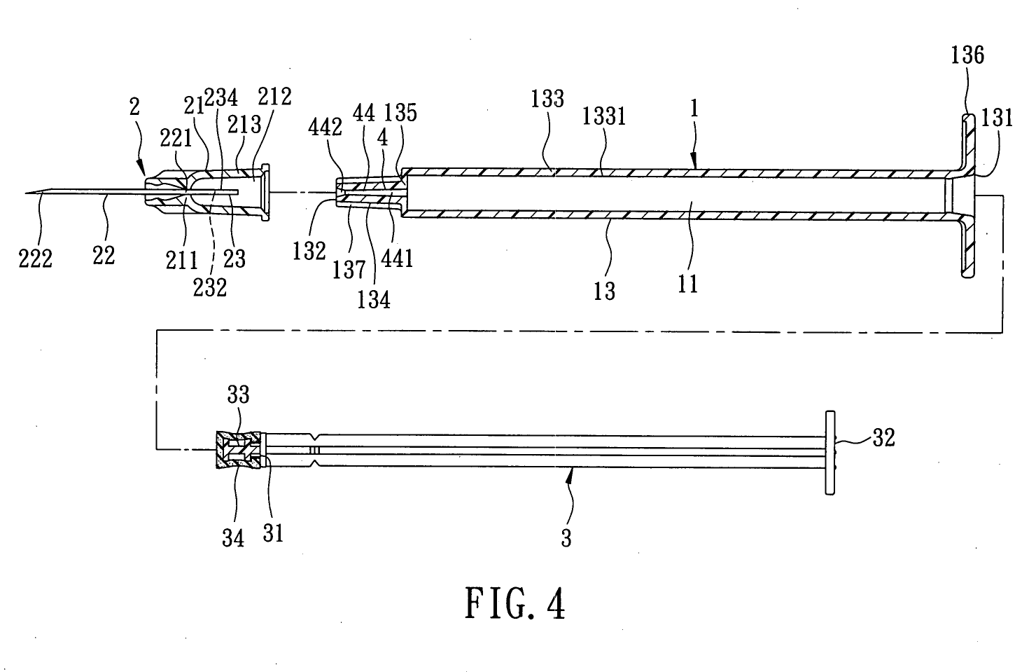

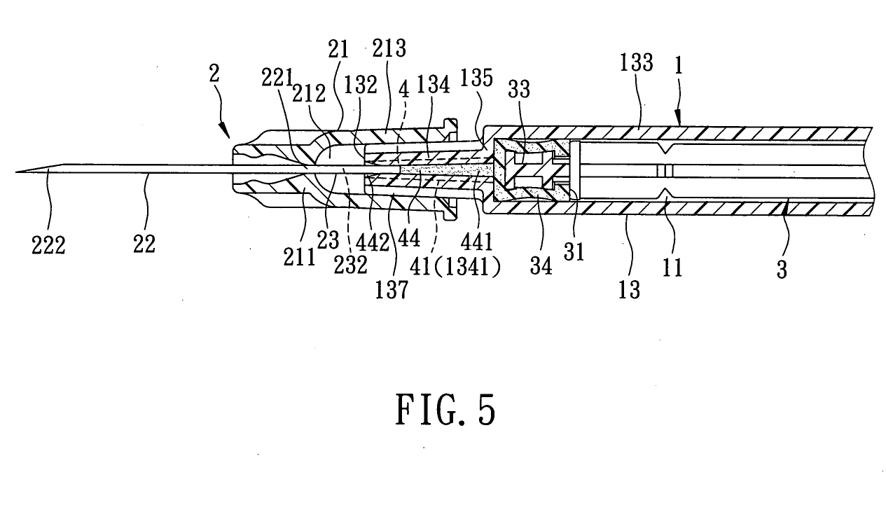

Referring to FIGS. 4 and 5, the second preferred embodiment of a disposable syringe according to this invention is similar to the first embodiment in construction. The difference resides in that the tubular abutment surface 41 of the space minimizing member 4 is integrally formed with the front inner wall surface 1341 of the small-diameter wall portion 134. That is, the space minimizing member 4 is integrally formed with the barrel 1 so as to narrow an inner space defined by the small-diameter wall portion 134 such that the small-diameter wall portion 134 has an inner diameter the same as that of the passageway 44 described above in the first embodiment. In addition, the small-diameter wall portion 134 has a plurality of ribs 137 which are angularly displaced from one another about the axis and each of which extends in the axial direction such that when the rear sleeve wall portion 213 of the needle seat 21 is sleeved fittingly on the ribs 137, the small-diameter wall portion 134 can be contracted to thereby reduce the inner diameter of the passageway 44. Thus, with the provision of the ribs 137, the small-diameter wall portion 134 can be made thinner.

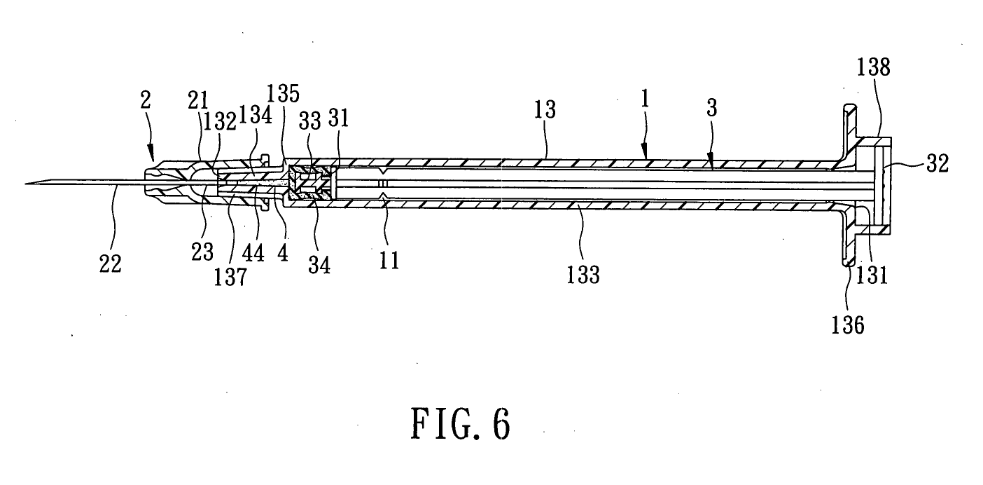

Referring to FIG. 6, the third preferred embodiment of a disposable syringe according to this invention is similar to the second embodiment in construction, except that the barrel 1 further has a tubular guard portion 138 which extends rearwardly from the finger flange 136 and which defines a receiving space communicated with the accommodation chamber 11 such that the thumb rest 32 of the plunger 3 extends rearwardly and outwardly of the tubular guard portion 138 for manual operation. The thumb rest 32 can be received in the receiving space when the plunger 3 reaches the shoulder 135 so as to prevent reuse of the syringe.

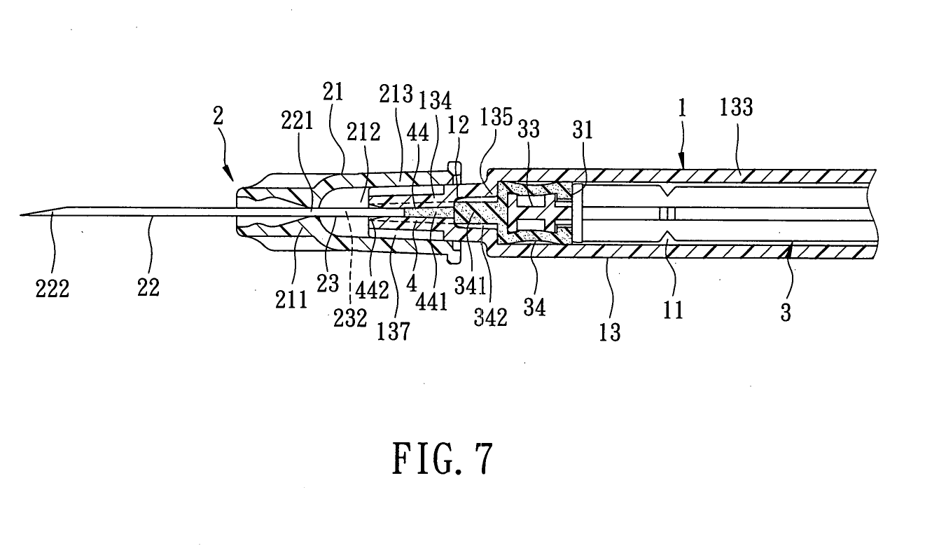

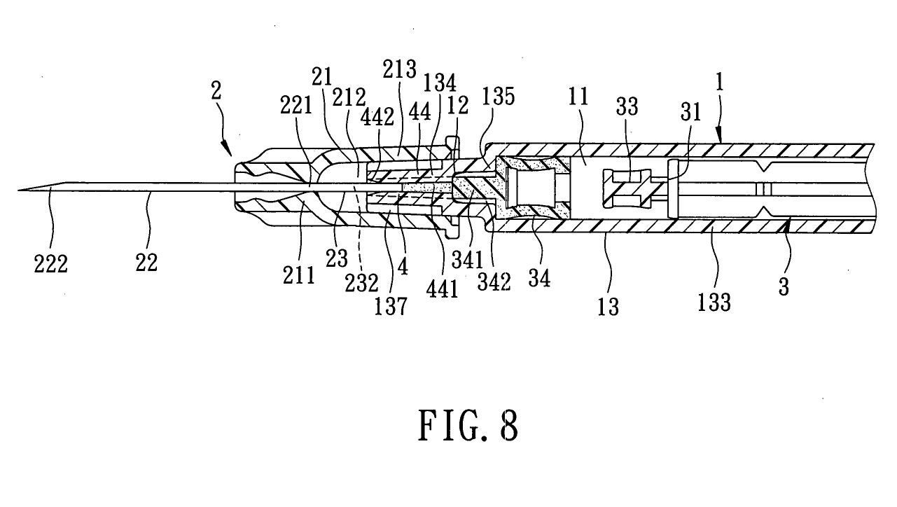

Referring to FIG. 7, the fourth preferred embodiment of a disposable syringe according to this invention is similar to the second embodiment in construction. In the fourth embodiment, the deformable sealing member 34 is detachably disposed on the carrier portion 33. In addition, an interrupting portion 341 extends from the sealing member 34 and is configured to be forced by the carrier portion 33 to plug the passage 12, thereby interrupting fluid communication between the accommodation chamber 11 and the passageway 44 and rendering the syringe unreusable. Thus, referring to FIG. 8, once the plunger 3 is pulled rearwards, the deformable sealing member 34 and the interrupting portion 341 are detached from the carrier portion 33 and are left behind in the passage 12, thereby preventing reuse of the syringe. A plurality of ribs 342 are formed on and surround the interrupting portion 341, and have a dimension larger than that of the passage 12 for ensuring retention of the interrupting portion 341 in the passage 12.

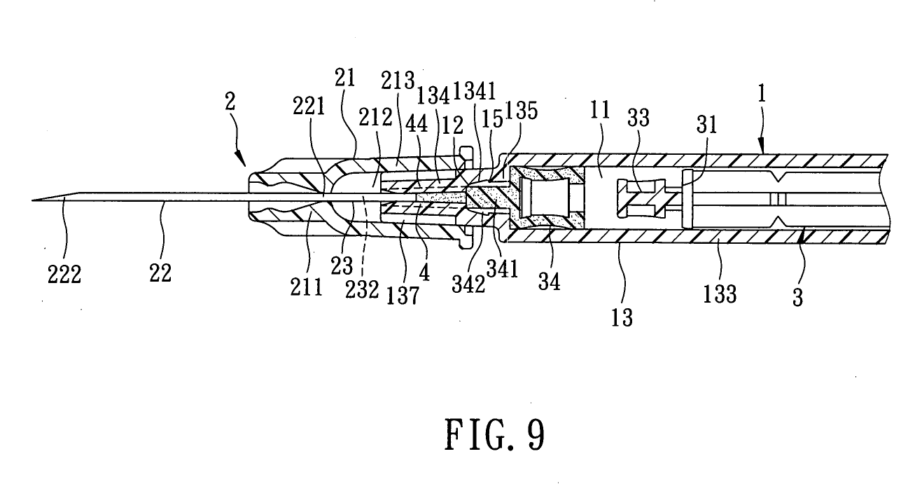

Referring to FIG. 9, the fifth preferred embodiment of a disposable syringe according to this invention is similar to the fourth embodiment in construction. In the fifth embodiment, the small-diameter wall portion 134 has an annular retaining ledge 15 which is disposed on the front inner wall surface 1341 to further ensure retention of the interrupting portion 341 in the passage 12.

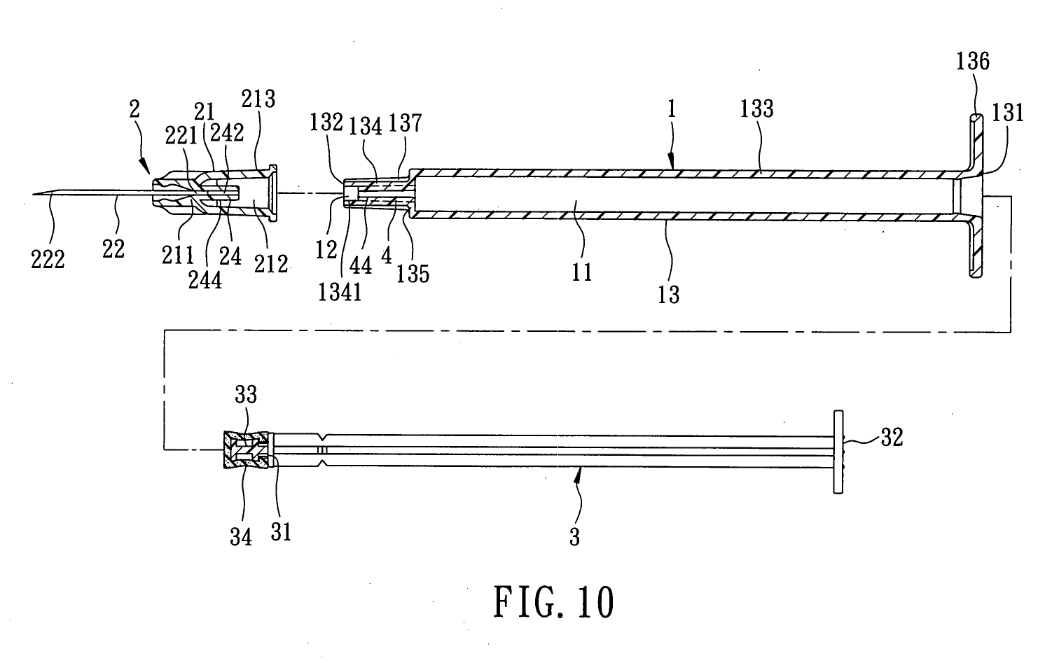

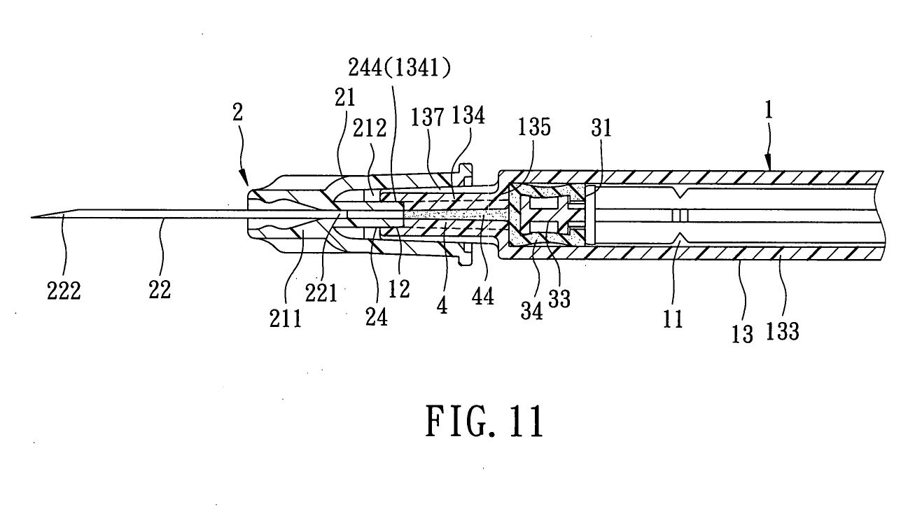

Referring to FIGS. 10 and 11, the sixth preferred embodiment of a disposable syringe according to this invention is similar to the second embodiment in construction. In the sixth embodiment, the tubular insert 24 is integrally formed with and extends rearwardly from the front retaining wall portion 211 of the needle seat 21. Thus, when the rear sleeve wall portion 213 is sleeved on the small-diameter wall portion 134, the tubular insert 24 is press-fitted into the passage 12 from the front barrel end 132 by virtue of a fluid-tight engagement between the front inner wall surface 1341 of the small-diameter wall portion 134 and the outer tubular surface 244 of the tubular insert 24.

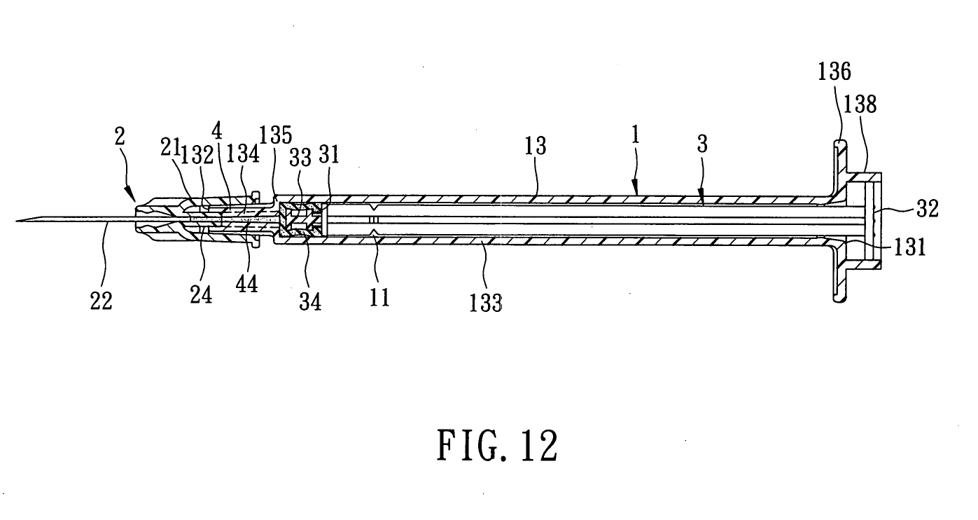

Referring to FIG. 12, the seventh preferred embodiment of a disposable syringe according to this invention is similar to the sixth embodiment in construction. This embodiment differs from the sixth embodiment mainly in that, as in the third embodiment, the barrel 1 further has a tubular guard portion 138 which extends rearwardly from the finger flange 136 and which defines a receiving space communicated with the accommodation chamber 11 such that the thumb rest 32 of the plunger 3 extends rearwardly and outwardly of the tubular guard portion 138 for manual operation. The thumb rest 32 can be received in the receiving space when the plunger 3 reaches the shoulder 135 so as to prevent reuse of the syringe.

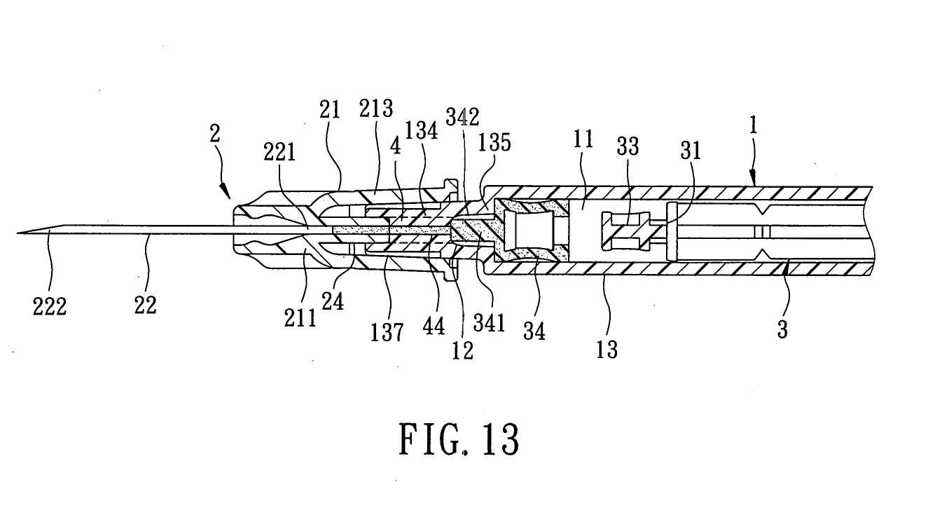

Referring to FIG. 13, the eighth preferred embodiment of a disposable syringe according to this invention is similar to the sixth embodiment in construction. This embodiment differs from the sixth embodiment mainly in that, as in the fourth embodiment, the deformable sealing member 34 is detachably disposed on the carrier portion 33, and has an interrupting portion 341 extending forwardly to be forced by the carrier portion 33 to plug the passage 12 in the small-diameter wall portion 134, thereby interrupting fluid communication between the accommodation chamber 11 and the passageway 44 and rendering the syringe unreusable. Thus, once the plunger 3 is pulled rearwards, the deformable sealing member 34 and the interrupting portion 341 are detached from the carrier portion 33 and are left behind in the passage 12, thereby preventing reuse of the syringe. A plurality of ribs 342 are formed on and surround the interrupting portion 341 for ensuring retention in the passage 12.

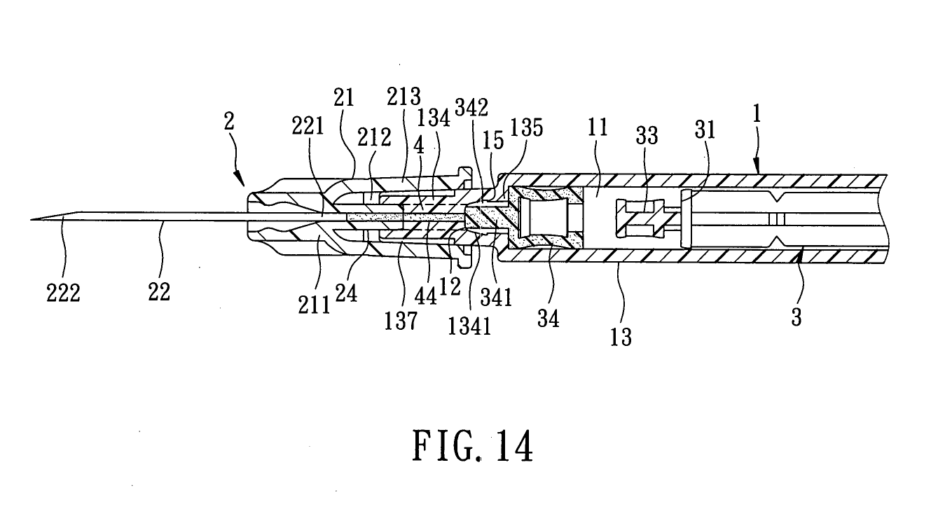

Further, referring to FIG. 14, the ninth preferred embodiment of a disposable syringe according to this invention is similar to the eighth embodiment in construction. This embodiment differs from the eighth embodiment mainly in that as in the fifth embodiment, the small-diameter wall portion 134 has an annular retaining ledge 15 which is disposed on the front inner wall surface 1341 to further ensure retention of the interrupting portion 341 in the passage 12.

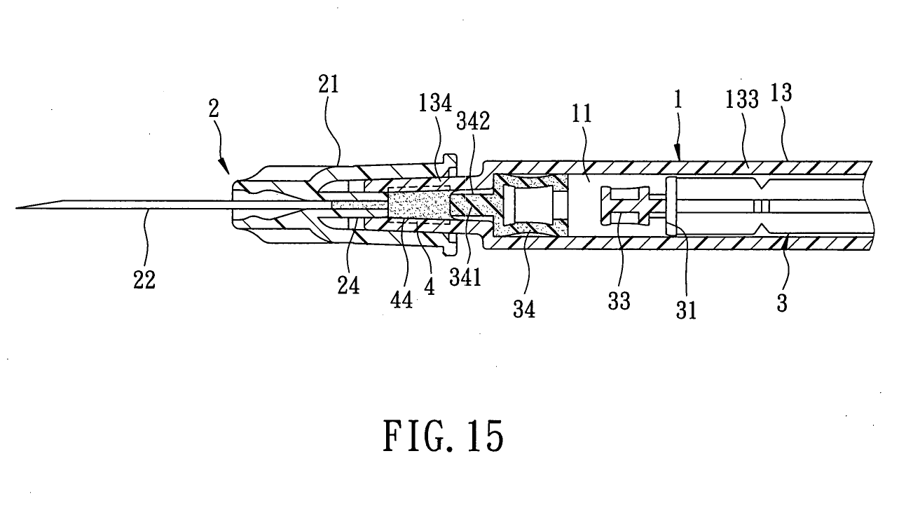

Referring to FIG. 15, the tenth preferred embodiment of a disposable syringe according to this invention is similar to the eighth embodiment in construction. In the tenth embodiment, the dimension of the passageway 44 and the passage 12 are the same such that the barrel 1 has an even inner diameter at the small-diameter wall portion 134 so as to facilitate fabrication of the barrel 1. In addition, the tubular insert 24 may be configured to have a larger outer diameter so as to be press-fitted into the passage 12, thereby facilitating fabrication of the tubular insert 24.

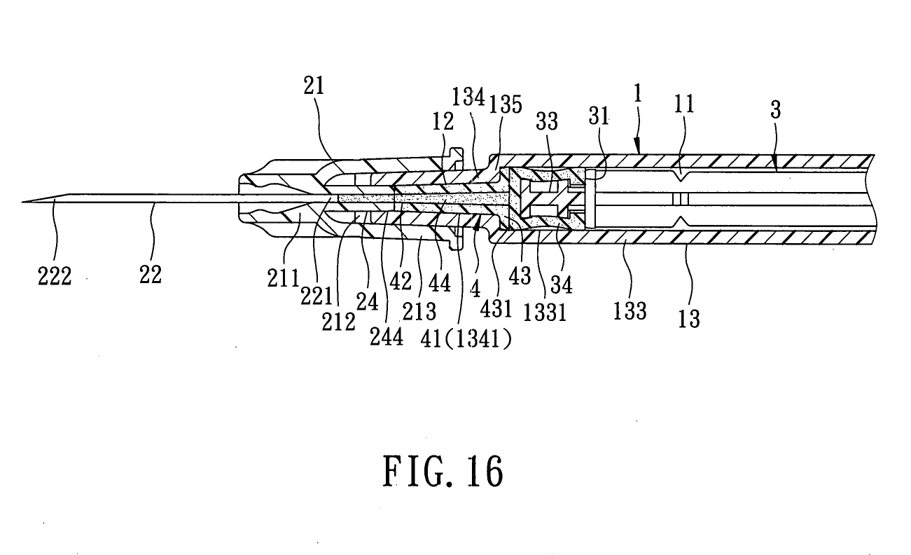

Referring to FIG. 16, the eleventh preferred embodiment of a disposable syringe according to this invention is similar to the sixth embodiment in construction. This embodiment differs from the sixth embodiment mainly in that as in the first embodiment, the space minimizing member 4 has a tubular abutment surface 41, a tubular grip surface 42 which defines a passageway 44, and a tubular rear barrier flange 43. The space minimizing member 4 is disposed to be inserted into the passage 12 in the small-diameter wall portion 134 from the rear barrel end 131 to bring the tubular abutment surface 41 into fluid-tight abutment against the front inner wall surface 1341, and to bring the tubular rear barrier flange 43 into abutment against the shoulder 135. The outer flange surface 431 of the flange 43 is in fluid-tight engagement with the rear inner wall surface 1331.

While the present invention has been described in connection with what is considered the most practical and preferred embodiments, it is understood that this invention is not limited to the disclosed embodiments but is intended to cover various arrangements included within the spirit and scope of the broadest interpretations and equivalent arrangements.

We claim:

1. A disposable syringe comprising:
a barrel including front and rear barrel ends opposite to each other along an axis in an axial direction, and a surrounding barrel wall which interconnects said front and rear barrel ends, and which includes
small-diameter and large-diameter wall portions that are disposed proximate to said front and rear barrel ends, respectively, and that respectively have front and rear inner wall surfaces defining a passage and an accommodation chamber which are in fluid communication with each other, and
a shoulder that is interposed between said small-diameter and large-diameter wall portions;
a needle cannula having a rear secured end and a front tip end opposite to each other along the axis;
a tubular needle seat having a front retaining wall portion which retains said rear secured end of said needle cannula to permit forward extension of said front tip end from said front retaining wall portion, and a rear sleeve wall portion which is opposite to said front retaining wall portion along the axis to define a duct that is fluidly communicated with said needle cannula, and which is detachably sleeved on said small-diameter wall portion to fluidly communicate said duct with said passage;
a tubular insert disposed to extend from said front retaining wall portion along the axis and into said duct, and having an inner tubular surface which defines a fluid channel that is disposed to be fluidly communicated with said rear secured end of said needle cannula, and an outer tubular surface which is opposite to said inner tubular surface in radial directions relative to the axis;
a space minimizing member including
a tubular abutment surface which surrounds the axis, and which is disposed to be in a fluid-tight engagement with said front inner wall surface, and
a tubular grip surface which is surrounded by said tubular abutment surface, and which defines a passageway that fluidly communicates said duct with said accommodation chamber such that when said rear sleeve wall portion is sleeved on said small-diameter wall portion, said tubular insert is inserted into said passage and is in a fluid-tight engagement with said tubular grip surface to thereby communicate said fluid channel with said accommodation chamber; and
a plunger which is disposed to be movable in said accommodation chamber of said large-diameter wall portion along the axis so as to force a medication fluid in said accommodation chamber to flow into said needle cannula through said passageway and said fluid channel.

2. The disposable syringe of claim 1, wherein said barrel further has a tubular guard portion which extends rearwardly from said rear barrel end and which defines a receiving space communicated with said accommodation chamber, said plunger having a thumb rest which extends rearwardly and outwardly of said tubular guard portion for manual operation, and which is received in said receiving space when said plunger reaches said shoulder.

3. The disposable syringe of claim 1, wherein said tubular abutment surface of said space minimizing member is integrally formed with said front inner wall surface of said small-diameter wall portion.

4. The disposable syringe of claim 3, wherein said plunger has a front end wall which confronts said passage, a carrier portion which extends from said front end wall forwardly along the axis, and a hollow deformable sealing member which is detachably disposed on said carrier portion and which is in fluid-tight and sliding engagement with said rear inner wall surface, and an interrupting portion which is engaged with said sealing member and which is configured to be forced by said carrier portion to plug said passage, thereby interrupting fluid communication between said accommodation chamber and said passageway and rendering said syringe unreusable.

5. The disposable syringe of claim 4, wherein said small-diameter wall portion has an annular retaining ledge disposed on said front inner wall surface to ensure retention of said interrupting portion in said passage.

6. The disposable syringe of claim 1, wherein said tubular insert is integrally formed with and extends rearwardly from said rear secured end of said needle cannula along the axis.

7. The disposable syringe of claim 6, wherein said tubular insert is configured such that when said rear sleeve wall portion is sleeved on said small-diameter wall portion, said tubular insert is press-fitted into said passageway by virtue of a fluid-tight engagement between said outer tubular surface and said tubular grip surface.

8. The disposable syringe of claim 1, wherein said tubular insert is integrally formed with said front retaining wall portion.

9. The disposable syringe of claim 8, wherein said tubular insert is configured such that when said rear sleeve wall portion is sleeved on said small-diameter wall portion, said tubular insert is press-fitted into said passage by virtue of a fluid-tight engagement between said front inner wall surface and said outer tubular surface.

10. The disposable syringe of claim 1, wherein said space minimizing member is disposed to be inserted into said passage from said rear barrel end to bring said tubular abutment surface into abutment against said front inner wall surface.

11. The disposable syringe of claim 10, wherein said space minimizing member has a tubular rear barrier flange which is disposed to abut against said shoulder so as to prevent forward movement of said space minimizing member, and which extends radially and outwardly to terminate at an outer flange surface, said outer flange surface being in a fluid-tight engagement with said rear inner wall surface.

12. The disposable syringe of claim 1, wherein said small-diameter wall portion is in form of a plurality of ribs which are angularly displaced from one another about the axis.
